(19) United States
(12) Patent Application Publication
(10) Pub. No.: US 2009/0099033 A1
(43) Pub. Date: Apr. 16, 2009

(54) In vitro screening and evolution of proteins

(76) Inventors: Jeffrey Rogers, San Diego, CA (US); Genevieve Hansen, San Diego, CA (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 11/890,345

(22) Filed: Aug. 6, 2007

Related U.S. Application Data
(62) Division of application No. 11/018,798, filed on Dec. 21, 2004, now abandoned.

(51) Int. Cl. C40B 30/04; C12Q 1/68

(52) U.S. Cl. 506/9; 435/6

(57) ABSTRACT

The present invention provides a composition which links genotype and phenotype and provides a method for in vitro protein evolution and screening using said composition. The invention also facilitates the identification and isolation of proteins with selected properties from large pools of proteins. The composition and method of the invention can be used with eukaryotic (both mammalian and plant) and prokaryotic translation systems.

### FIELD OF THE INVENTION

The invention relates generally to the field of molecular biology, more particularly, this invention relates to compositions and methods for the identification and isolation of nucleic acids that encode a protein having desired properties from large pools of nucleic acids. The invention also relates to a composition and method that allows the principles of in vitro selection and in vitro evolution to be applied to proteins.

### BACKGROUND OF THE INVENTION

In vitro protein evolution methods require access to a highly varied population of test molecules, a way to select members of the population that exhibit the desired properties, and the ability to amplify the selected molecules with mutated variations to obtain another highly varied population for subsequent selection. For efficient protein evolution to occur it is necessary to have a means of producing and selecting from very large libraries. There currently exist several in vitro and in vivo methods for the evolution of proteins by amplifying and mutating the nucleic acids that encode the protein and selecting molecules out of populations of mutated nucleic acids that have desired properties. The in vivo methods involve screening small libraries <10⁸ molecules) but have the advantages of chaperones and the cellular environment. The in vitro methods allowing the screening of large libraries (>10¹³ molecules) but protein folding and disulfide bond formation can be problematic.

Examples of in vivo expression libraries include yeast two- or three-hybrid, yeast display and phage display methods. In vivo protein evolution methods suffer from various disadvantages, including a limited library size and relatively cumbersome screening steps. The limited library size is a significant limitation because the number of possible peptide sequences encoding a 10 residue sequence is 10¹³ and the majority of in vivo libraries are capable of the display of fewer than 10⁸ molecules. Therefore, the size of the libraries which can potentially be produced can exceed by several orders of magnitude the ability of current in vivo technologies to display all members of such library. Additionally, undesired selective pressures can be placed on the generation of variants by cellular constraints of the host.

In vitro expression libraries may use either prokaryotic or eukaryotic translation systems and typically rely upon ribosome display. These methods can link the protein and its encoding mRNA with the ribosome such that the entire complex is screened against a ligand of choice. Once the appropriate ribosome complex has been identified they are disrupted and the released mRNA is recovered and used to construct cDNA. Three critical parts of the ribosome display process are (i) the stalling of the ribosome to produce stable complexes (for example by addition of cyclohexamide, rifampicin, or chloramphenicol or the deletion of a stop codon), (ii) the screening of the attached protein for its interaction with ligand which may be interfered with by the large size of the ribosome in comparison to the protein, and (iii) the recovery of the mRNA (e.g., Hanes et al. (1997) Proc. Natl. Acad. Sci., USA, 94:4937, WO 98/54312, WO 99/11777).

A recently developed variation of ribosome display is to attach the protein to its coding sequence during translation by using ribosomal peptidyl transferase with puromycin attached to a linker DNA (e.g., Roberts et al. (1997) Proc. Natl. Acad. Sci., USA, 94:12297, Wilson et al. (2001) Proc. Natl. Acad. Sci., USA, 98:3750, U.S. Pat. No. 6,261,804, U.S. Pat. No. 6,416,950, WO 01/90414). Once the coding sequence and the peptide are linked, the ribosome is dissociated prior to screening the protein RNA fusion for interaction with its ligand. Due to the covalent nature of the puromycin linkage between the mRNA and the protein it encodes, selection experiments are not limited to the extremely mild conditions that must be used for the ribosome display approaches that involve non-covalent complex formation.

The mild conditions necessary for ribosome display and the technical difficulty of mRNA display are shortcomings of these methods that would be useful to address in an alternative in vitro protein selection method. There is also a need for a method that will provide for the robust linking of an mRNA to the protein it encodes that may be used in the screening of proteins that bind other molecules or the screening of proteins that catalyze reactions. Neither ribosome display nor mRNA display are useful to screen proteins that catalyze reactions. It would also be useful to identify a method for the linking of genotype-phenotype and the selection of favorable proteins in a single step of the method. In ribosome display and mRNA display this linking of genotype and phenotype and the selection of proteins is a two-step process within the method that may result in a high background level of mRNA selection.

Throughout this application, various publications are referenced by author and date. The disclosures of these publications in their entireties are hereby incorporated by reference into this application in order to more fully describe the state of the art as known to those skilled therein as of the date of the invention described and claimed herein.

### SUMMARY OF THE INVENTION

This invention is directed to the selection of nucleic acids and polypeptides and provides a composition for the linking of genotype and phenotype and methods for in vitro protein evolution and screening using said composition.

In one aspect, the invention provides a composition that is referred to herein as a “SBP/DNA chimera”. An “SBP/DNA chimera” comprises an oligodeoxyribonucleotide that is covalently linked to a selective binding partner (the “SBP”) as is discussed more fully in the following. The oligodeoxyribonucleotide may also be referred to as a DNA oligonucleotide. The oligodeoxyribonucleotide comprises a primer sequence and a linker sequence. The primer sequence is the 3′ portion of the oligodeoxyribonucleotide and its function in the method of the invention is to hybridize to the mRNA that is used in the method of the invention thus providing a link between the SBP/DNA chimera and each mRNA in the population which is being screened. The linker sequence is of any desired length and can be a cleavable sequence, for example a single stranded sequence that is sensitive to DNase. The selective binding partner of the SBP/DNA chimera is any suitable molecule, including without limitation a protein, peptide, amino acid, nucleic acid, small molecule, hormone and carbohydrate. An amino acid can be phosphorylated or non-phosphorylated. A nucleic acid can be single stranded or double stranded.

In another aspect, the invention provides a composition having a first and a second component. The first component is the SBP/DNA chimera and the second component is an RNA comprising: a translation initiation site; a start codon; a nucleotide sequence encoding a protein of interest; and, a primer binding site. The SBP/DNA chimera binds to the RNA by hybridization of the primer sequence of the SBP/DNA chimera to the primer binding site of the RNA. The selective binding partner of the SBP IDNA chimera is bound by or binds to the protein of interest that is encoded by the RNA.

In one embodiment, the binding affinity of the selective binding partner to the protein of interest varies with the amino acid sequence of the protein of interest.

In another embodiment the RNA includes a tag sequence encoding a polypeptide. The tag sequence is located on the RNA molecule either prior to or after the sequence encoding the protein of interest and when the RN A is translated the encoded polypeptide will be fused to the protein of interest.

In a further embodiment, the RNA includes a sequence encoding a linker. The sequence encoding the linker is located after the sequence encoding the protein of interest and when the RNA is translated will be fused to the protein of interest. The linker functions to allow the nascent protein to exit the ribosome following translation such that the protein is free from the constraint of the ribosome and folds properly.

In another embodiment, the protein of interest is an immunologically active molecule and the selective binding partner is an antigen or epitope for such molecule. In another embodiment the protein of interest is a nucleic acid binding protein and the selective binding partner is a nucleic acid. In another embodiment the protein of interest is a carbohydrate binding protein and the selective binding partner is a carbohydrate. In another embodiment the protein of interest is an enzyme and the selective binding partner is a substrate.

In yet another embodiment the selective binding partner is attached to a solid substrate. The selective binding partner is attached directly to the solid substrate or is attached to the solid substrate via a linker.

In another aspect, the invention provides a method for selecting a nucleic acid molecule that encodes a protein of interest. In this method, the first and second components described above are obtained and the first component is bound to the second component by hybridizing the primer sequence to the primer binding site. The RNA sequence is translated to produce a protein of interest under conditions that allow the protein of interest to bind with the selective binding partner of the SBP/DNA chimera. A complex of the protein of interest bound to the SBP/DNA chimera which is bound to the RNA sequence encoding said protein is produced by the hybridization between the primer sequence and the primer binding site (a “nascent protein-SBP/DNA chimera-RNA complex”). The nascent protein-SBP/DNA chimera-RNA complex is isolated. The linker portion of the SBP/DNA chimera is then cleaved, and the RNA sequence is separated from the nascent protein.

In another aspect, the invention includes a method for selecting a nucleic acid molecule that encodes a protein of interest. In this method, the first and second components described above are obtained, and the RNA of the second component includes a tag sequence. The first component is bound to the second component by hybridizing the primer sequence to the primer binding site. The RNA sequence is translated to produce a protein of interest under conditions that allow the protein of interest to bind with the selective binding partner of the SBP/DNA chimera, thereby producing a complex of the protein of interest bound to the SBP/DNA chimera which is bound to the RNA sequence encoding the protein by the hybridization between the primer sequence and the primer binding site (a “nascent protein-SBP/DNA chimera-RNA complex”). The nascent protein-SBP/DNA chimera-RNA complex is isolated using a binding partner for the polypeptide encoded by the tag sequence. The linker portion of the SBP/DNA chimera is cleaved, and the RNA sequence is separated from the nascent protein.

In another aspect, the invention provides a method for selecting a nucleic acid molecule. In this method, the first and second components described above are obtained and the first component is bound to the second component by hybridizing the primer sequence to the primer binding site. The RNA sequence and included tag sequence are translated to produce a protein of interest and the polypeptide encoded by the tag sequence under conditions that allow the polypeptide encoded by the tag sequence to bind with the selective binding partner of the SBP/DNA chimera, thereby producing a complex of the protein of interest bound to the SBP/DNA chimera which is bound to the RNA sequence encoding the protein by the hybridization between the primer sequence and the primer binding site (a “nascent protein-SBP/DNA chimera-RNA complex”). The nascent protein-SBP/DNA chimera-RNA complex is isolated using a binding partner for the protein of interest. The linker portion of the SBP IDNA chimera is cleaved, and the RNA sequence is separated from the nascent protein.

The invention also provides for any of the methods described above to be repeated using the RNA sequence that was isolated in the final step of the method in order to modify the identified RNA and select for a nucleic acid that encodes a protein of interest. In one embodiment the isolated RNA sequence is altered prior to repeating the steps of the method. In another embodiment the RNA sequence is amplified by reverse transcribing the RNA prior to repeating the steps of the method of the invention. In a specific embodiment the primer binding sequence of the SBP/DNA chimera is the primer for reverse transcription of the RNA.

The invention also provides for the nascent protein-SBP/DNA chimera-RNA complex to be isolated using a binding partner for the polypeptide that is encoded by the tag sequence and is present in the nascent protein.

In another embodiment of the invention, the selective binding partner binds to the polypeptide encoded by the tag sequence, and upon translation of the RNA sequence and the tag sequence the protein of interest and the polypeptide are produced under conditions that allow the polypeptide encoded by the tag sequence to bind with said selective binding partner, thereby producing a complex of the polypeptide encoded by the tag sequence bound to the selective binding partner which is bound to the RNA sequence encoding said protein by the hybridization between the primer sequence and the primer binding site.

In one embodiment, this nascent protein-SBP/DNA chimera-RNA complex is isolated using a binding partner for the protein of interest. In another embodiment, the binding partner is linked to a solid substrate, either directly or through a linker.

As used herein “selective binding partner” includes any molecule that has a specific, covalent or non-covalent, affinity for the protein of interest or for a polypeptide encoded by a tag sequence which molecule is a part of the SBP/DNA chimera. Such a selective binding partner is, without limitation, a protein, peptide, antibody, amino acid (including phosphorylated and non-phosphorylated amino acids), small molecule, hormone, carbohydrate or nucleic acid. By a “binding partner” is meant any molecule which may be useful as a selective binding partner, but is not a part of the SBP/DNA chimera. A selective binding partner or a binding partner may optionally be attached to a solid support.

As used herein “SBP/DNA chimera” includes a DNA oligonucleotide that is covalently bonded to a selective binding partner. The DNA oligonucleotide is comprised of a 3′ primer sequence and a linker. The linker can be a cleavable sequence, e.g., a single-stranded DNase sensitive region. The linker may be of any desired length including without limitation, S, 10, 20, 50, 100 or more than 100 nucleotides.

As used herein a “tag sequence” means a nucleic acid that encodes a polypeptide sequence which is translated as a part of the mRNA. This encoded polypeptide sequence is a sequence of amino acids that are recognized and bound by a binding partner that is distinct from the selective binding partner. For example, the tag sequence can encode the FLAG epitope (DYKDDDDK, SEQ ID NO:1) that is specifically bound by an anti-FLAG antibody. Alternatively, the tag sequence encodes a c-Myc epitope (EQKLISEEDL SEQ ID NO:2) that is specifically bound by an anti-c-Myc antibody or a His epitope (HHHHHH, SEQ ID NO:3) that is specifically bound by an anti-His antibody.

By a “protein” is meant any two or more naturally occurring or modified amino acids joined by one or more peptide bonds. “Protein,” “polypeptide” and “peptide” are used interchangeably herein.

As used herein a “nucleic acid” means any two or more covalently bonded nucleotides or nucleotide analogs or derivatives. This term includes, without limitation, DNA, RNA, PNA, and combinations thereof. A “nucleic acid coding sequence” can therefore be DNA (for example, cDNA), RNA, PNA, or a combination thereof. By “DNA” is meant a sequence of two or more covalently bonded, naturally occurring or modified deoxyribonucleotides. By “RNA” is meant a sequence of two or more covalently bonded, naturally occurring or modified ribonucleotides. One example of a modified RNA included within this term is phosphorothioate RNA.

By “linked” is meant covalently or non-covalently associated. By “covalently bonded” is meant that a selective binding partner is joined to a DNA oligonucleotide either directly through a covalent bond or indirectly through another covalently bonded sequence. By “non-covalently bonded” is meant joined together by means other than a covalent bond (for example, by hybridization or Van der Waals interaction).

As used herein a “population” means a group of more than one molecule (for example, more than one RNA, DNA, or RNA-protein fusion molecule). Because the methods of the invention facilitate selections which begin, if desired, with large numbers of candidate molecules, a “population” according to the invention can mean, for example, more than 10⁹, 10¹⁰, 10¹¹, 10¹² or 10¹³ molecules. When present in such a population of molecules, a desired protein may be selected horn other members of the population.

By “selecting” is meant substantially partitioning a molecule horn other molecules in a population. For example, a “selecting” step provides at least a 2-fold, a 30-fold, a 100a-fold, or a 1000-fold enrichment of a desired molecule relative to undesired molecules in a population following the selection step. Each disclosed method of the invention for selecting a nucleic acid molecule step may be repeated any number of times, and combinations of the methods of the invention may be used.

The term “translation initiation sequence” is used herein to mean any sequence which is capable of providing a site for ribosome binding and the efficient initiation of translation. In bacterial systems, this region is sometimes referred to as a Shine-Delgarno sequence.

The term “strong promoter for in vitro transcription” is used herein to mean any sequence for which RNA polymerase has a high binding affinity and is useful for the initiation of in vitro transcription of mRNA, such as the T7 promoter.

By “start codon” is meant three bases which signal the beginning of a protein coding sequence. Generally these bases or AUG (or A TG); however, any other base triplet capable of being utilized in this manner may be substituted.

The term “solid support” means any substrate to which a nucleic acid molecule or protein can be bound, such as, a column (or column material), bead, test tube, microtiter dish, solid particle (for example, agarose or sepharose), microchip (for example, silicon, silicon-glass, or gold chip), or membrane (for example, the membrane of a liposome or vesicle) to which an affinity complex may be bound, either directly or indirectly (for example, through other binding partner intermediates such as other antibodies or Protein A), or in which an affinity complex may be embedded (for example, through a receptor or channel).

Other features and advantages of the invention will be apparent from the following detailed description, and from the claims.

### BRIEF DESCRIPTION OF THE DRAWINGS

FIG. 1 is a schematic representation of a method of the invention.

FIG. 2 is a schematic representation of a construct useful for the preparation of DNA libraries which libraries are useful in the method of the invention.

FIG. 3 is a representation of a Coomassie blue stained polyacrylamide gel showing the results of fast protein liquid chromatography (“FPLC”) purification of a SBP/DNA chimera containing lysozyme as the selective binding partner, which SBP/DNA chimera can be used in the method of the invention. M=marker, F1 and F2=fraction I and fraction 2 that were collected from the FPLC after elution using 0.5 M NaCl. Lys=lysozyme alone. The location of the lysozyme-DNA chimera and lysozyme alone are indicated to the left of the figure.

FIGS. 4a and 4b are representations of West em blots that depict the functionality of in vitro translated proteins. Rabbit reticulocyte lysate translated constructs (V_{HH} plus or V_{HH} minus) were bound to either lysozyme-agarose beads (FIG. 4a) or FLAG agarose beads (FIG. 4b). P=in vitro translated protein before incubation with beads and E=protein eluted after incubation and washing of beads. M=marker.

### DETAILED DESCRIPTION

The purpose of the present invention is to allow the principles of in vitro selection and in vitro evolution to be applied to proteins. The present invention facilitates the isolation of nucleic acids encoding proteins with desired properties from large pools of nucleic acids. This invention solves the problem of recovering and amplifying the nucleic acid encoding a desired protein sequence by the provision of a composition, the SBP/DNA chimera, which hybridizes to the mRNA coding sequence and is selectively bound by the protein encoded by such mRNA. The composition and method of the invention can be used with eukaryotic (both mammalian and plant) and prokaryotic translation systems.

The present invention provides a composition for the linking of phenotype and genotype, and an improved method of in vitro identification of proteins and protein evolution. The composition serves two purposes, first to halt translation and secondly to serve as a link between the nascent protein and its mRNA. The composition and method of the invention can also be utilized with mRNA which includes a stop codon in which event the composition of the invention does not halt translation as that will occur naturally, but serves to link the nascent protein and its mRNA.

The described composition and methods offer at least two advantages over the existing in vitro technology for directed protein evolution and protein screening (e.g., Roberts et al. (1997) Froc. Natl. Acad. Sci. 94:12297; Hanes et al. (1997) Froc. Natl. Acad. Sci. 94:4937). First, the composition and method of the invention can be used to link a protein of interest to its mRNA in the presence of a stop codon at the end of its mRNA. A polypeptide encodes by the tag sequence at the N-terminal end of the nascent protein binds to an SBP/DNA chimera that includes a selective binding protein for the polypeptide encoded by a tag sequence before a stop codon is reached. Once a stop codon is reached, the ribosome dissociates and the nascent protein remains attached to the mRNA that directed its synthesis via the interactions of the nascent protein or the polypeptide encoded by the tag sequence with the SBP/DNA chimera. Another advantage of the present method is its ability to evolve not only proteins that bind to things, but proteins having catalytic activity as well. The target substrate is attached to the mRNA of any successfully evolved catalyst through the SBP/DNA chimera. In other selection schemes, there is no method to identify which proteins are able to modify a substrate. In the method of the invention, proximity between the nascent protein and its target (and kinetics) identify which proteins are capable of catalysis as the RNA hybridized to a catalyzed substrate encodes the desired protein. The composition and method of the invention are further illustrated below.

An SBP/DNA chimera can be routinely made and purified quickly and effectively. The method of its construction is mild, e.g., neutral pH, physiological salt, and temperatures from 4° C. to room temperature or above, allowing any protein chosen as the selective binding partner to remain in a native state, and produces SBP/DNA chimeras that include several sizes and charges of selective binding partners (e.g., BSA, lysozyme and antibodies) that are linked to an oligodeoxyribonucleotide.

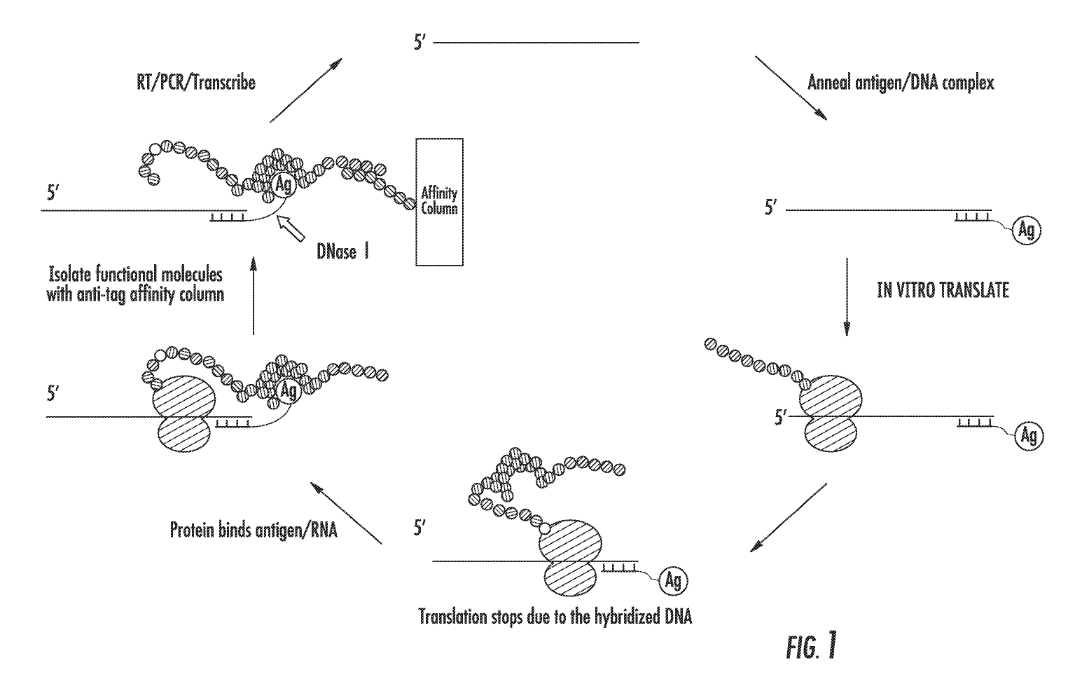

The composition and method of the invention are shown schematically in FIG. 1. The SBP IDN A chimera of the invention may be designed for use with any protein of interest or may be designed to select for proteins that associate with the selective binding partner when no such protein has previously been identified. To form the chimera, an amino terminated oligodeoxyribonucleotide is conjugated to the selective binding partner.

The oligodeoxyribonucleotide portion of the SBP/DNA chimera comprises a primer sequence and linker sequence. The linker is covalently bonded to the selective binding partner. The primer sequence is the 3′ portion of said oligodeoxyribonucleotide which hybridizes to the 3′ end of the population of mRNA prepared from the library to be screened. The linker is an additional 5′ portion of oligodeoxyribonucleotide that is of any desired length, for example, 10, 20, 30, 40, 50 or more deoxyribonucleotides, and it functions to provide a spacer between the selective binding partner and the 3′ primer sequence. The linker portion of the DNA is optionally designed to be cleavable, for example, it may contain a single stranded DNase susceptible region such that the 3′ primer portion of the oligodeoxyribonucleotide can be released from the selective binding partner. The linker portion is of any desired length. For example, in a non-limiting embodiment described herein, a 20 base-pair single stranded DNAse-I susceptible region was utilized as the linker. The linker thus provides a unique cleavage site for DNaseI to separate the mRNA from the protein of interest, and the 3′ primer portion serves as a primer for reverse transcriptase such that the mRNA can be amplified for further study.

If the selective binding partner is small enough (e.g., having a molecular weight of less than about 1000 daltons), it may be directly coupled to a 5′ NHS-ester-terminated oligonucleotide. A low molecular weight SBP is incubated with the solid support (e.g. a column) for an hour at room temperature. The support is then washed several times with, e.g., acetonitrile. The SBP/DNA chimera is then deprotected and purified as with a normal DNA oligonucleotide. A Selective binding partner can be commercially obtained, covalently linked to an oligonucleotide, and purified on a solid support. For selective binding partners that are at least about 1000 daltons, the selective binding partner is covalently bound to the DNA through a 5′ primary amino group of the DNA, with the use of the cross-linking reagent disuccinimidyl suberate (“DSS”, available, e.g., from Pierce, Rockford, Ill.).

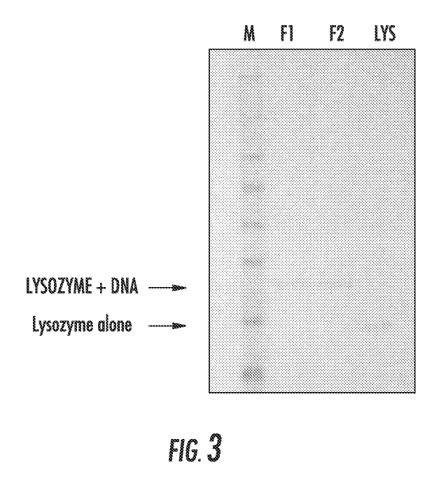

The DSS method involves three basic steps. The first step is the reaction of the 5′-amino terminated oligonucleotide with DSS and the subsequent termination of this reaction before the non-reacted end of the DSS hydrolyzes. This is accomplished by reacting an excess of DSS with the 5′-amino terminated oligonucleotide for 30 seconds and then quenching the reaction by gel filtration in the presence of 1 mM NaOAc, pH 4 at 4° C. The second step is the conjugation of the DSS-activated oligonucleotide with the selective binding partner at pH 8.5. The selective binding partner is present in excess in this reaction and the reaction is allowed to go to completion overnight at room temperature. The third step is the separation and isolation of the various reaction products. This is achieved through FPLC utilizing an ion exchange column and a NaCl gradient. Gel filtration, high performance liquid chromatography (“HPLC”) or gel extraction (or any combination of the above) may also be used to purify DNA-SBP conjugates. Unreacted selective binding partner elutes below 0.2 M NaCl. The SBP/DNA chimera elutes just before the DNA oligonucleotide alone, at approximately 0.5 M NaCl. The various fractions are tested for SBP, ability to prime reverse transcription reactions, and successful SBP IDNA chimera conjugation (e.g., by Coomassie-stained protein gel analysis). SBP/DNA chimeras typically run slower than selective binding partners alone (FIG. 3).

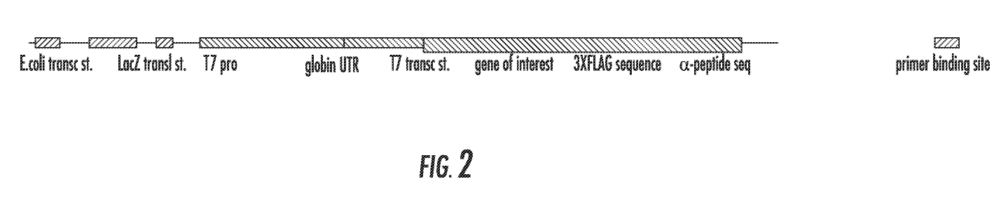

A DNA library that is used according to the method of the invention can be prepared and transcribed as discussed below. A DNA library may be comprised of either a single gene, which may be mutated through any known method such as error prone PCR, or a population of genes. A schematic representation of a sample construct is provided in FIG. 2 and is discussed in greater detail below. Each construct contains a strong promoter for in vitro transcription, a 5′ untranslated region (“UTR”), a strong eukaryotic translation initiation sequence, the coding sequence of the gene, a linker sequence, and a 3′ terminal DNA primer binding site. The function of the linker sequence is to allow the translated protein of interest to exit the ribosome, fold properly, and recognize its target. The construct may also contain a tag sequence which sequence may be located upstream or downstream of the gene of interest. The primers used to make this library are the same regardless of the library being constructed. Any new library (e.g., comprising either a population of genes or a population of mutated forms of a target gene) may be amplified with tagged oligodoexyribonucleotides that overlap with the above-mentioned primers. The DNA population is transcribed with an RNA polymerase that recognizes the promoter for in vitro transcription for 60 minutes at 37° C., followed by DNase I treatment for an additional 15 minutes at 37° C. The RNA is then purified using known techniques, such as a Qiagen™ kit (Qiagen, Valencia, Calif.) or ethanol precipitation. The DNase I step ensures that the only DNA amplified during the amplification step will originate from reverse transcribed mRNA that was selected. The 5′ primer site for PCR is the first 20 bases of the mRNA transcript. This primer is 50 bases long and restores the strong promoter sequence. The 3′ primer binds to the primer binding site of the target-DNA chimera and has a T_{M} of greater than 60° C.

The following steps of the method of the invention are illustrated in FIG. 1 and are discussed below using a protein which binds to a selective binding partner as the example. The illustrated steps begin at the top center of FIG. 1 and progress clockwise around the schematic representation.

The mRNA is prepared from a DNA library as described above. The mRNA is combined with the SBP/DNA chimera to allow the 3′ primer sequence of the SBP/DNA chimera to hybridize to the 3′ binding sequence of the mRNA. The mRNA is then translated with an in vitro translation system. In the method of the invention the SBPIDNA chimera that is hybridized to the mRNA serves three main functions: (1) it causes the ribosome to pause and translation to stop; (2) it presents the selective binding partner for interaction with the expressed protein and creates the genotype/phenotype link for only such proteins that interact with the selective binding partner; and (3) it provides a cleavable link between the nascent protein and mRNA. The complex of nascent protein SBP/DNA chimera-mRNA is then separated, for example as discussed below, from the other molecules present in the translation reactions. After selection nascent protein and mRNA are decoupled by DNaseI. The mRNA is isolated by any known technique and is amplified by reverse transcription followed by amplification using PCR.

The following is a more detailed example of the method of the invention using protein which binds to a selective binding partner as the example. The prepared mRNA is combined with the SBP/DNA chimera as mentioned above mRNA is then translated. After 10 minutes of translation at 30° C., the reaction is supplemented with magnesium (50 mM final concentration). The reaction is incubated on ice for 5 minutes to allow nascent protein/selective binding partner interaction. 100 mM EDT A is then added to dissociate the ribosome complex. Only the mRNAs that encodes a protein that binds to the selective binding partner will still be a part of a complex (consisting of the nascent protein-SBP/DNA chimera-mRNA). The nascent protein is bound to the selective binding partner and the 3′ binding sequence of the mRNA is hybridized to the 3′ primer sequence of the oligodeoxyribonucleotide.

The translation reaction, including complexes of the nascent protein-SBP/DNA chimera-mRNA, is then diluted 10 fold in washing buffer (50 mM Tris, pH 8, 150 mM NaCl, 0.1% Tween 20, 5 mM EDTA, 0.1 mg/mL BSA) and added to a 96-well plate preincubated with the binding partner for the polypeptide encoded by the tag sequence (e.g., if FLAG is the polypeptide encoded by the tag sequence then anti-FLAG antibody is used). The reaction is incubated at room temperature for one hour. The well is then washed at least 15 times with wash buffer, and then the mRNA is eluted by the addition of DNase I.

In the event that the mRNA is prepared from a cDNA library, such that the mRNA contains a stop codon prior to the 3′ terminal primer binding site to which the SBP/DNA chimera hybridizes, the selective binding partner of the SBP/DNA chimera is a binding partner for the polypeptide encoded by a tag sequence which has been inserted into the mRNA prior to the coding sequence of the gene. Thus, the polypeptide encoded by the tag sequence binds to or is bound by the selective binding partner present in the SBP/DNA chimera before the stop codon is reached. When the stop codon of the mRNA is reached, the protein is already linked to its mRNA coding sequence through the interaction of the polypeptide encoded by the tag sequence and the selective binding partner and a complex of the nascent protein-SBP/DNA chimera-mRNA is formed. The complex of the nascent protein-SBP/DNA chimera-mRNA is separated from other protein/chimera complexes in the pool through use of an affinity column for the nascent protein of interest as generally discussed below.

Other tag sequences can be used instead of the sequence which encodes the FLAG polypeptide, such as a tag sequence that encodes a histidine epitope or a c-Myc epitope. The tag sequence can also be a novel sequence selected using the method of the invention. Also, instead of a selection on 96-well plates, an affinity column can be prepared (i.e., by linking an protein that binds to the polypeptide encoded by a tag sequence to CNBr-activated Sepharose 4B (Amersham, Piscataway, N.J.>> and the nascent protein-RNA complexes are separated ITom the other components of the in vitro translation reaction by purification over the prepared affinity column. The protein-RNA complexes can also be separated from the other components of the in vitro translation reaction by purification based on size, e.g., centrifugation sedimentation rates, or by size exclusion chromatography.

When a cDNA library is used, the mRNA is linked to its nascent protein through the interaction of the polypeptide encoded by the tag sequence and the SBP/DNA chimera. The complex of the nascent proteins-SBP/DNA chimera-mRNA is then incubated with a solid-support-bound binding partner of the protein of interest (prepared as above). Only mRNAs which have produced functional proteins are chosen, as they are the only RNA molecules attached to SBPIDNA chimera molecules that have a functional nascent protein that binds to the solid-support-bound binding partner. For screening of enzymes, the SBP/DNA chimera may optionally be conjugated to a solid support through the selective binding partners. This is done in situations where the nascent protein is an enzyme that cleaves a molecule present between the solid support and the DNA of the SBP/DNA chimera (i.e., a peptide cleaved by a protease).

The separation of wanted/unwanted sequences is accomplished through the linking of genotype and phenotype usually in conditions of nascent protein/selective binding partner recognition. This interaction is very tight, as dissociation can result in the loss of the genotype/phenotype link. For example, a typical antibody binds its target with at least low nM affinity. At 4° C. this provides a connection that lasts 2 hours. Molecules with higher dissociation constants require the selection to be done faster to be successful. Temperature can also be used to select extremely tight binding molecules as the reaction can be done at elevated temperatures. Only molecules that can remain attached during high temperature incubations will be selected.

The mRNA of the complex of the nascent protein-SBP/DNA chimera-mRNA are then eluted from the complex for use in further rounds of selection. The linker portion of the oligodeoxyribonucleotide of the SBP/DNA chimera is cleaved by Dnase I and the released mRNA is purified, for example, by use of a Qiagen™ kit (Qiagen, Valencia, Calif.). The linker portion is cleaved, for example, by the addition of DNase I in the event that a DNase sensitive region was included in such an oligodeoxyribonucleotide. As the only DNA molecules in the complex are present in the SBP/DNA chimera, this allows for a very gentle elution of the mRNA molecules of interest. The eluted RNAs are reverse transcribed at 45° C., and then PCR amplified with primers which restore the strong transcription promoter. This PCR amplification can be mutagenic (0.7-10%) or non mutagenic, e.g., as described by Cadwell & Joyce, PCR Methods Appl., 1992, August; 2(1):28-33 and Vartanian et. al, Nucleic Acids Res., (1996), 24(14):2627-31. The resultant double-stranded DNA molecules are ready for transcription in a second round of in vitro selection using the method of the invention.

### Uses of the Method of the Invention

The method of the invention can be used to identify and/or select for antibodies or proteins having binding affinity for a desired selective binding partner and can be accomplished through either random mutagenesis of an antibody or protein of interest or the screening of a cDNA library. It is useful for an antibody to have a single chain format. However, other proteins can also be utilized as potential protein molecules that bind other molecules “non-antibody binders”). The ideal protein for use in the method of the invention is small, contains no cysteines, and is very stable. A non-limiting example for an alternative antibody scaffold is the B 1 fragment from protein G. Proteins and antibodies having binding affinity for a selective binding partner can be used in nonsystemic therapeutic applications (oral, topical or nasal delivery), diagnostic applications (protein chips, Westerns) or as knock-out tools inside model organisms. Non-human proteins can be used therapeutically by delivery methods other than by systemic delivery. Selective binding partners can also be used on protein chips to identify and quantify amounts of various proteins of interest. Additionally, non-antibody binders are useful in vivo as “intrabodies”, and can be targeted against various cellular proteins to disrupt interactions and determine protein function.

The usefulness of the method of the invention to for the screening of cDNA libraries provides several functional genomics applications. For example, as the selective binding partner can be a protein, small molecule, RNA, or dsDNA, unknown targets of small molecule drugs or antibiotics can be screened for using the method of the invention. Screening the cDNA of an organism that is known to be affected by a small molecules using the method of the invention, may result in the isolation of proteins that interact with that small molecule, which proteins may then be further studied.

The method of the invention can also be used to screen for proteins that bind RNA or sugars. For example, the method of the invention can be used to identify and selectively evolve transcription factors for specific RNA binding targets, and to identify and selectively evolve proteins which can bind or modify cellularly important sugars. There are also several industrially important sugar modifying enzymes which can be discovered or improved using the method of the invention.

The potential applications of the method of the invention are used in the field of metabolomics and the development of a small molecule chip. Once a protein that can bind a small molecule has been identified, this protein can be evolved to select for specific binding abilities. In addition such a protein is useful for the detection of the small-molecule to which it binds. The detection of the small molecule by the protein that binds to it could be accomplished, e.g. through ELISA sandwich methods or by using the method of the invention to evolve a modified version of the protein that binds its small molecule target only in the presence of a dye. For example, the method of the invention has been used to evolve a protein that binds to its small molecule target only in the presence of a dye, and a chip could be made containing such protein and the presence of the target molecule is determined by fluorescence of the dye upon binding of the protein to the target molecule.

Also, the method of the invention can be used to screen several molecules at once or even a library of molecules (known as “target multiplexing”). Target multiplexing rapidly increases the rate of discovery and also permits the screening of complex mixtures of targets such as whole cells. Instead of hybridizing one target-SBP to the 3′ end of the mRNA, a mixture of 50-100 target-SBPs can be annealed to the 3′ end of the mRNA. The resulting selected proteins are then further screened using the method of the invention and each target individually to determine which of the target(s) are bound by which protein(s).

By using a target substrate as the selective binding partner to use the method of the invention can be used to link genotype and phenotype of an enzyme as well. For ample, if the protein of interest is a protease, then an SBP/DNA chimera is designed such that the selective binding partner is a target substrate which is attached to a solid support such as a column, and the mRNA is then hybridized to the primer sequence of the SBP/DNA chimera as described for the method of the invention. A successfully transcribed and folded protease cleaves the target substrate, thus releasing the mRNA from the solid support and separating it from the other RNA molecules. The method of the invention also allows the evolution of proteases to screen for substrate specificity, increased stability or kinetics. In a similar manner, ligases are screened for their ability to add a tag to their target substrate, and kinases are screened for their ability to phosphorylate/dephosphorylate a target substrate.

There are several examples in nature of small molecules that allosterically effect proteins. It has also been shown in the RNAIDNA in vitro selection field, that allostery can be evolved into molecules. The method of the invention is used to add allosteric control to proteins of interest. This is helpful, e.g., for antibody fragments on protein chips that can be designed to fluoresce when they bind their targets. In this case, the protein target alters the shape of the antibody, allowing it to bind a dye. Antibodies are used to intracellularly to knock-out genes. Often when genes are knocked out in a model system (i.e., mice) the organism dies. Therefore, the role of the gene can never be ascertained. If an allosteric antibody is made using the method of the invention that is inactive until the addition of a small molecule turns it on, then chip profiling effects of lethal genes and the almost real-time monitoring of the knock-out effects can be observed. The antibody that is constitutively expressed can be introduced into a host organism using standard techniques and will remain inactive until a small molecule effector is added. The small molecule activates the antibody and the antibody binds its target. The effects of the antibody binding its target is monitored visually, selectively, or through RNA profiling experiments.

### Exemplification.

An SBP/DNA chimera was prepared according to the method of the invention, and as described below, using lysozyme as the selective binding partner and an oligodeoxyribonucleotide of 47 bases. The 3′ primer portion of the oligodeoxyribonucleotide was 27 bases and the 5′ linker portion of the oligodeoxyribonucleotide was 20 bases. The SBP/DNA chimera was purified by FPLC using an anion exchange column and resulted in a unique SBP/DNA chimera peak and pure lysozyme compound. 10 uL of samples were incubated at 9SOc for 2 minutes in SDS loading buffer. Samples were then run on a 4-12% polyacrylamide gel for 30 minutes, followed by Coomassie blue staining. FIG. 3 shows two fractions (F1 and F2) that were eluted from the FPLC just before the DNA-alone peak. The location of the lysozyme-DNA chimera and lysozyme alone are indicated to the left of the figure.

Specifically, the method of the invention was conducted using the camel antilysozyme VHH gene as the gene of interest (Ghahroudi, et al., Febs Letters, 414 (1997) 521-526). The camel anti-lysozyme V_{HH} gene was constructed by PCR in six overlapping pieces, the sequences of which are provided in SEQ ID NOS:4-9, and Table 1 below using oligonucleotide primers that were 100% identical to the portion to be cloned and were 50-100 nucleotides in length.

TABLE 1

| | | |
|---|---|---|
| SEQ ID | | AACCATGGACGTTCAGCTGCAGGCTTCTGGT |
| NO: 4 | | GGTGGTTCTGTTCAGGCTGGTGGTTCTCTGC |
| | | GTCTGTCTTGCGCTGCTTCTGGTTACACCAT |
| | | CGGTCCGTACTGC |
| SEQ IO | (contains | TTTACCCGGAGCCTGACGGAACCAACCCAT |
| NO: 5 | amino acid | GCAGTACGGACCGAT |
| | position 37 | |
| | shown in | |
| | bold-faced | |
| | type are in | |
| | reverse com- | |
| | plement | |
| | orientation) | |
| SEQ 10 | (contains | CAGGCTCCGGGTAAAGAACGTGAAGGTGTT |
| NO: 6 | amino acid | GCTGCTATCAACATGGG |
| | positions 44, | |
| | 45 and 47 | |
| | shown in | |
| | bold-faced | |
| | type) | |
| SEQ 10 | | GCAGGTAAACGGTGTTTTTAGCGTTGTCCTG |
| NO: 7 | | AGAGATGGTGAAACGACCTTTAACAGAGTCA |
| | | GCGTAGTAGGTGATACCACCACCCATGTTGA |
| | | TAGCAG |
| SEQ 10 | | CACCGTTTACCTGCTGATGAACTCTCTGGAA |
| NO: 8 | | CCGGAAGACACCGCTATCTACTACTGCGCTG |
| | | CTGACTCTACCATCTACGCTTCTTACTACGA |
| | | ATGCGGT |
| SEQ 10 | | TTGCTAGCAGAAGAAACGGTAACCTGGGTAC |
| NO: 9 | | CCTGACCCCAAGAGTCGTAACCGTAACCACC |
| | | GGTAGACAGACCGTGACCGCATTCGTAGTAA |

The six overlapping oligodeoxyribonucleotides were combined in a PCR reaction and 40 cycles of PCR were undertaken. Conditions were standard PCR conditions, with a T_{M} of 60 degrees Celsius and an extension time of 30 seconds. 0.5 μM total of all six primers were added. The PCR reaction was then diluted 100 fold in a new PCR reaction with only 5′ and 3′ external primers (which primers contained restriction sites). After 20 cycles of PCR, the PCR construct was cloned into a plasmid to confirm the in vivo production of the protein product. Namely, the V_{HH} gene construct was cloned into a modified pT7Blue-2 vector (Novagen, Madison, Wis.) that allows transcription both in vitro and in E. coli.

The pT7Blue-2 vector was modified to contain the 3XFLAG peptide sequence upstream of the a-peptide fragment of the p-galactosidase gene. The 3XFLAG sequence was constructed by two overlapping DNA oligodeoxyribonucleotides. These were extended using Taq polymerase and then the product was amplified using PCR primers with regions that overlapped with the vector construct. A PCR product of the 5′ end of the vector and a PCR product of the 3′ end of the vector were then combined with the 3XFLAG PCR product. These three PCR products were PCR amplified together to give a full-length product with a 3XFLAG sequence inserted before the a-peptide fragments. The FLAG epitope was utilized as it permits easy detection of the produced protein and also provides a purification moiety to which column purification of the nascent proteinSBP/DNA chimera-mRNA complex is possible.

The V_{HH} gene was then cloned upstream of the 3XFLAG sequence, but downstream of the transcription initiation site, the UTR and the translational initiation site. This was accomplished through the generation of overlapping PCR fragments (as above). The resulting construct is schematically represented on FIG. 2. The globin UTR in the construct functions to prevent secondary structure in the RNA near the translational start site. The α-peptide fragment of the β-galactosidase gene functions as a spacer/linker to permit newly made protein of the gene of interest to exit the ribosome and correctly fold. This spacer/linker is long enough for the gene of interest protein to fold properly, but not too long to encourage intermolecular interactions instead of intramolecular reactions. The 3′ primer binding site is the RNA sequence where the 3′ primer sequence present in the SBP/DNA chimera binds. The hybridization of the mRNA to the oligodeoxyribonucleotide functions to pause the ribosome and connects genotype to phenotype in successful gene of interest variants.

Ligated plasmids of the above described construct were initially transformed into DH5a competent cells (Invitrogen, Carlsbad, Calif.) by the method recommended by the manufacturer. Then plasmid was isolated by Qiagen plasmid purification kits (Qiagen, Valencia, Calif.) and retransformed with NovaBlue E. coli competent cells (Novagen, Madison, Wis.) by the method recommended by the manufacturer. This was done because of the high transformation efficiency of DH5α, which allows the transformation and amplification of ligated plasmids. This ensures there will be enough material to transform the lower efficiency NovaBlue cells. All cells were grown in Luria-Broth supplemented with 100 μg/mL of carbenicillin (Sigma, St. Louis, Mo.) at 37° C. Protein production was induced with the addition of 1 mM IPTG during late log phase to confirm the production of functional V HH from the construct. Once the functional production of V_{HH} from the construct was confirmed by gel analysis and Biacore (Piscataway, N.J.) analysis, the construct was translated in vitro to demonstrate the method of the invention.

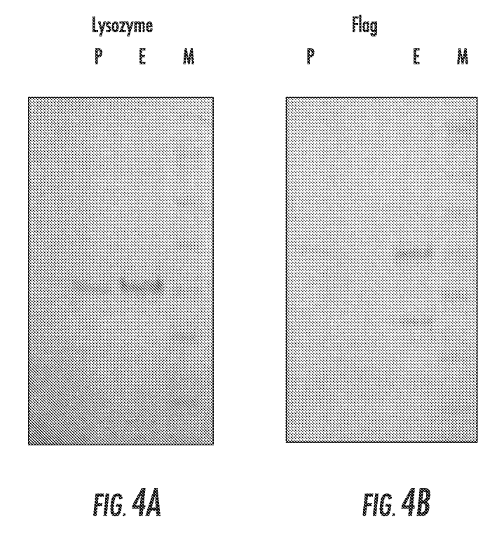

In vitro translation of the V_{HH} construct using the reticulocyte lysate IVT kit (Ambion, Austin, Tex.) results in functional V HH antibody being isolated. Two constructs (one containing the lysozyme-V_{HH} and one lacking the lysozyme-V_{HH}) were co-translated in one tube and then the tube was divided into two equal parts. One part was incubated with anti-FLAG agarose and one part was incubated with lysozyme-agarose. After one hour with shaking, the samples were washed five times with PBS+0.1% Tween 20 and eluted with the addition of 8M urea at 95° C. for 2 minutes. The samples were run on a 4-12% polyacrylamide gel and then the proteins were transferred to a nitrocellulose membrane. Subsequent blocking (5% Milk-PBS-O.1% Tween 20) and staining anti-FLAG alkaline phosphatase) resulted in the representations of the Western blots shown in FIGS. 4a and 4b. The in vitro molecules both bind to anti-FLAG agarose (the tag sequence, but only the V_{HH} containing construct binds to lysozyme-agarose beads (through the action of V_{HH} binding). FIG. 4a shows that the in the mixture of V_{HH} containing and non-V_{HH} containing in vitro translated protein, the larger V_{HH} containing fragment is enriched when the mixture is mixed with lysozyme-agarose beads. FIG. 4b shows that both the V_{HH} and non-V_{HH} proteins are maintained by the anti-FLAG agarose (shown in FIG. 4b, lane E). Sample “P” on FIGS. 4a and 4b show what the samples looked like before incubation with either agarose samples. Sample “E” on FIGS. 4a and 4b shows the protein that is eluted after incubation with lysozyme-agarose beads and washing.

Three constructs have been made to test and optimize the proposed in vitro selection protocol: (1) a construct lacking any insert (FLAG and linker sequence); (2) a construct containing the anti-lysozyme V_{HH} antibody; and (3) a construct containing the anti-IgG domain B1 from protein G. These constructs were made and are screened according to the method of the invention against a number of positive and negative selective binding partners (BSA, anti-flag antibody, lysozyme and mouse IgG to permit optimization of incubation times, reaction conditions and washing buffers.

The invention described herein uses in vitro techniques to add enzyme screening and cDNA library screening to the list of things that non-compartmentalized in vitro selection systems can accomplish.

While the invention has been described in connection with specific embodiments thereof, it will be understood that it is capable of further modifications and this application is intended to cover any variations, uses or adaptations of the composition and method of the invention following, in general, the principles of the invention and including such departures ITom the present disclosure that come within known or customary practice within the art to which the invention pertains and may be applied to the essential features hereinbefore set forth.

1-16. (canceled)

17. A method for selecting a nucleic acid molecule that encodes a protein of interest, comprising:
a) obtaining a population of first components comprising:
a translation initiation site;
a start codon;
an RNA sequence encoding a protein, the RNA sequence varying for different first components in said population; and
a primer binding site; and
b) obtaining a second component comprising:
a DNA primer sequence,
a linker, and
a selective binding partner that binds to a protein of interest, the binding affinity of the binding partner for the protein of interest varying with the amino acid sequence of the protein of interest;
c) hybridizing the primer sequence to the primer binding site to bind said first component to said second component;
d) translating said RNA sequence to produce said protein under conditions that allow a protein comprising a protein of interest to bind with said selective binding partner thereby producing a complex of the protein of interest bound to the selective binding partner which is bound to the RNA sequence encoding said protein by the hybridization between the primer sequence and the primer binding site;
e) isolating said complex of step (d);
f) cleaving said linker of said second construct; and
g) isolating said RNA sequence that encodes said protein of interest, thereby selecting a nucleic acid molecule that encodes a protein of interest.

18. The method of claim 17, further comprising repeating steps (a) through (g) using said isolated RNA sequence obtained in step (g) at least once whereby.

19. The method of claim 18, further comprising altering the sequence of said RNA sequence encoding said protein of interest between repetitions of steps (a) through (g).

20. The method of claim 17, further comprising reverse transcribing said RNA sequence into a DNA sequence.

21. The method of claim 20, wherein said reverse transcription uses said DNA primer sequence of said second component.

22. The method of claim 17, wherein the linker of the second component is a cleavable linker.

23. The method of claim 17, wherein the selective binding partner is selected from the group consisting of a protein, peptide, phosphorylated or non-phosphorylated amino acid, nucleic acid, carbohydrate, small molecule, hormone, and carbohydrate.

24. The method of claim 17, wherein said first component further comprises a tag sequence.

25. The method of claim 24, wherein said tag sequence is selected from the group consisting of a nucleic acid encoding the FLAG epitope, a nucleic acid encoding a c-Mycepitope, and a nucleic acid encoding a His epitope.

26. The method of claim 17, wherein the protein of interest is an immunologically active molecule, and the selective binding partner is an antigen or epitope.

27. The method of claim 17, wherein the protein of interest is a nucleic acid binding protein, and the selective binding partner is a nucleic acid.

28. The method of claim 17, wherein the protein of interest is a carbohydrate binding protein, and the selective binding partner is a carbohydrate.

29. The method of claim 17, wherein said selective binding partner is further attached to a solid substrate.

30. The method of claim 17, further comprising a linker between said selective binding partner and said solid substrate.

31. The method of claim 17, wherein said population of first components is obtained from a DNA library.

32. A method for selecting a nucleic acid molecule that encodes a protein of interest comprising:
a) obtaining a population of first components comprising:
a translation initiation site;
a start codon;
a tag sequence;
an RNA sequence encoding a protein, said RNA sequence varying for different first components in said population; and
a primer binding site; and
b) obtaining a second component comprising:
a DNA primer sequence;
a linker; and
a selective binding partner that binds to the polypeptide encoded by said tag sequence of said first component
c) hybridizing the primer sequence to the primer binding site to bind said first component to said second component;
d) translating said RNA sequence to produce said protein under conditions that allow said polypeptide encoded by said tag sequence to bind with said selective binding partner thereby producing a complex of the protein bound to the RNA sequence encoding said protein by binding of the polypeptide encoded by the tag sequence to the selective binding partner and by the hybridization between the primer sequence and the primer binding site;
e) isolating said complex of step (d) using a binding partner for a protein of interest under conditions that allow a protein comprising a protein of interest to bind with said binding partner thereby isolating a complex of step (d) comprising an RNA sequence encoding a protein of interest;
f) cleaving said linker of said second construct; and
g) isolating said RNA sequence that encodes said protein of interest thereby selecting a nucleic acid molecule that encodes a protein of interest.

33. A method for selecting a nucleic acid molecule that encodes a protein of interest, comprising:
a) obtaining a first component comprising
a DNA primer sequence,
a linker, and
a selective binding partner that binds to a protein or tag sequence;
b) obtaining a population of second components comprising
a translation initiation site
a 5′ untranslated region,
a start codon,
a tag sequence,
an RNA sequence encoding a protein wherein said RNA sequence varies for different second components in said population, and a primer binding site;
c) hybridizing the primer sequence to the primer binding site to bind said first component to said second component;
d) translating said RNA sequence to produce said protein under conditions that allow a protein comprising a protein of interest to bind with said selective binding partner, thereby producing a complex of a protein of interest bound to the RNA sequence encoding said protein through the binding of the protein of interest to the selective binding partner, and through the hybridization of the primer sequence to the primer binding site;
e) isolating said complex of step (d) using a solid support comprising a binding partner directed against a polypeptide encoded by the tag sequence;
f) cleaving said linker of said second component; and
g) isolating said RNA sequence that encodes said protein of interest thereby selecting a nucleic acid molecule that encodes a protein of interest.


SEQUENCE LISTING (9 sequences)

SEQ ID NO: 1 - protein, 8 aa, Artificial Sequence
  Chemically synthesized
DYKDDDDK

SEQ ID NO: 2 - protein, 10 aa, Artificial Sequence
  Chemically synthesized
EQKLISEEDL

SEQ ID NO: 3 - protein, 6 aa, Artificial Sequence
  Chemically synthesized
HHHHHH

SEQ ID NO: 4 - DNA, 106 bp, Artificial Sequence
  Chemically synthesized
aaccatggacgttcagctgcaggcttctggtggtggttctgttcaggctggtggttctctgcgtctgtcttgcgctgcttctggttacaccatcggtccgtactgc

SEQ ID NO: 5 - DNA, 45 bp, Artificial Sequence
  Chemically synthesized
tttacccggagcctgacggaaccaacccatgcagtacggaccgat

SEQ ID NO: 6 - DNA, 47 bp, Artificial Sequence
  Chemically synthesized
caggctccgggtaaagaacgtgaaggtgttgctgctatcaacatggg

SEQ ID NO: 7 - DNA, 99 bp, Artificial Sequence
  Chemically synthesized
gcaggtaaacggtgtttttagcgttgtcctgagagatggtgaaacgacctttaacagagtcagcgtagtaggtgataccaccacccatgttgatagcag

SEQ ID NO: 8 - DNA, 100 bp, Artificial Sequence
  Chemically synthesized
caccgtttacctgctgatgaactctctggaaccggaagacaccgctatctactactgcgctgctgactctaccatctacgcttcttactacgaatgcggt

SEQ ID NO: 9 - DNA, 93 bp, Artificial Sequence
  Chemically synthesized
ttgctagcagaagaaacggtaacctgggtaccctgaccccaagagtcgtaaccgtaaccaccggtagacagaccgtgaccgcattcgtagtaa